(19) United States
(12) Patent Application Publication
(10) Pub. No.: US 2022/0087003 A1
(43) Pub. Date: Mar. 17, 2022

(54) ELECTRODE ARRANGEMENT AND PLASMA SOURCE FOR GENERATING A NON-THERMAL PLASMA, AS WELL AS METHOD FOR OPERATING A PLASMA SOURCE

(71) Applicant: terraplasma GmbH, Garching (DE)

(72) Inventors: Maximilian Cantzler, Ebersberg (DE); Hannes Weilemann, München (DE)

(21) Appl. No.: 17/425,313

(22) PCT Filed: Jan. 25, 2019
(86) PCT No.: PCT/EP2019/051851

§ 371 Date: Jul. 22, 2021

(51) Int. Cl. H05H 1/24; A61L 2/00; A61L 2/26

(52) U.S. Cl. CPC H05H 1/2418; A61L 2/0011; A61L 2/26; A61N 1/44; H05H 2245/34; A61L 2202/11; H05H 2240/20

(57) ABSTRACT

The invention relates to an electrode arrangement for generating a non-thermal plasma, with: a first electrode and a second electrode, wherein the first electrode and the second electrode are electrically insulated from each other and spaced from each other by a dielectric element, characterized in that the second electrode has an Electroless Nickel Immersion Gold (ENIG) coating, or an Electroless Nickel Electroless Palladium Immersion Gold (ENEPIG) coating, or an Electroless Nickel Immersion Palladium Immersion Gold (ENIPIG) coating, or an Electroless Palladium (EP) coating, or an Electroless Palladium Immersion Gold (EPIG) coating, and/or the dielectric element is made of a woven glass reinforced hydrocarbon ceramic.

The invention relates to an electrode arrangement and plasma source for generating a non-thermal plasma, as well as a method for operating a plasma source.

Electrode arrangements for generating a non-thermal plasma typically have a first electrode and a second electrode, wherein the electrodes are electrically insulated and especially separated from each other by a dielectric element. Typical uses for such electrode arrangements, or the non-thermal plasmas generated by such electrode arrangements, are found in the fields of disinfection or sterilization, surface functionalization, as well as the medical field, in particular wound disinfection, wound treatment and healing, treating skin irritations, as well as treating bacterial, viral and fungal skin deceases. Another possible application is to refresh textiles and/or clothing instead or in addition of washing them.

To refresh the textiles and/or clothes different methods are possible:

One possibility is to mask malodor (by a suitable better odor or perfume) but this does not remove the odor molecules or the source of the malodor.

Remove the source of malodor (e.g. bacteria), but this does not remove existing odor molecules, but stops addition of new malodor (provided that all bacteria are permanently inactivated). Due to the fact that bacteria double their numbers in preferred zones such as an armpit in typically 5 minutes, accordingly a 3-log reduction to a thousandth ( 1/1000) is replenished after only 1 hour, so that an antibacterial method has to be repeated frequently.

Destroy malodor molecules by chemical action, in particular ozone is used, but due to its toxicity it has to be filtered out again from the air after the chemical oxidation of malodor molecules has been conducted. In addition, the reaction is slow and requires long interaction times (ozone molecules move thermally at only roughly 200 meters per second).

Removal of malodor molecules and sources by washing, wherein this is a standard process, which is partly mechanical and partly chemical. This works well in many circumstances, but it takes time, is costly, has a high (energy) carbon footprint and is generally an effort requiring access to a washing machine—which is not always possible (e.g. on travel). Another problem is that not all textiles or clothing can be washed, because they get deteriorated or even destroyed during washing. Furthermore, washing below 40° C. does not remove malodor sources (bacteria) and it may even enhance their growth.

For removal of malodor by dry cleaning, basically the same arguments as for washing apply. In addition dry cleaning agents, in particular chemical agents, also may affect some textiles and/or clothing negatively.

Conventional electrode arrangements can only be operated efficiently at comparably high voltage amplitudes, and therefore, for reasons of electrical safety, it is very difficult to move them close enough towards the surfaces to be treated, such as the skin of a human being. Moreover, conventional electrode arrangements are designed comparatively large and in particular rigid, which stands in the way of a miniaturization of devices that possess such an electrode arrangement, as well as a geometrically flexible use of the electrode arrangement.

The object of the invention is to create an electrode arrangement, as well as a plasma source for generating non-thermal plasma and a method for operating a plasma source, wherein the aforementioned disadvantages do not occur.

The object is achieved by creating the subject-matter of the independent claims. Advantageous embodiments will become apparent from the dependent claims.

The object is achieved in particular by creating an electrode arrangement for generating a non-thermal plasma, in particular for refreshing of surfaces, porous materials and fabrics, with a first electrode and a second electrode, wherein the first electrode and the second electrode are electrically insulated from each other and spaced from each other by a dielectric element, characterized in that the second electrode has an Electroless Nickel Immersion Gold (ENIG) coating, or an Electroless Nickel Electroless Palladium Immersion Gold (ENEPIG) coating, or an Electroless Nickel Immersion Palladium Immersion Gold (ENIPIG) coating, or an Electroless Palladium (EP) coating, or an Electroless Palladium Immersion Gold (EPIG) coating, and/or the dielectric element is made of a woven glass reinforced hydrocarbon ceramic. It is particularly preferred that the second electrode can be brought into contact with surfaces, porous materials and fabrics to be treated and with a non-thermal plasma. Hereby, the advantage can be obtained that the electrode arrangement allows the production of a copious plasma at room temperature and atmospheric pressure with a peak-to-peak voltage less than 5 kV. Furthermore, the electrode arrangement possesses a low abrasion electrode material (facing the surface of the textile and/or clothes to be treated) that does not discolor. Additionally, an electrode arrangement is provided that allows electrons to make direct contact with the treated surface and penetrate through the treated surface, if it is porous or fibrous (e.g. textile or clothing).

The electrode arrangement is in particular configured for generating surface microdischarges.

If a difference in potential, in particular an alternating voltage is applied to the two electrodes, surface microdischarges are formed on an active surface of the electrode arrangement that in turn cause a non-thermal plasma to be generated in the region of the active surface.

For this, the first electrode and second electrode are in particular designed as power electrodes.

The first electrode and second electrode are preferably arranged superimposed, i.e., in particular on two planes offset from each other, or are spaced from each other by the dielectric element, in particular in the form of a sandwich-like stack. Accordingly, an electric field can be generated by a voltage perpendicular across a surface of the dielectric when a difference in potential is applied between the two electrodes.

In connection with the present invention, the term “dielectric element” is to be understood as meaning a sheet-like non-conducting layer of given length, width and thickness, separating the first and second electrode from each other and defining the overall size of the electrode arrangement.

In connection with the present invention, the term “first electrode” is to be understood as meaning a uniform preferably sheet-like conducting element of given length, width and thickness.

In connection with the present invention, the term “second electrode” is to be understood as meaning a structured conducting element preferably consisting of at least one line of a given length, width and thickness. Furthermore the lines can be for example unidirectional, parallel, curvy or segmented (comb-like). Preferably, a combined length of all lines of the at least one line is larger than the width or thickness of the at least one line.

Furthermore in an alternative embodiment, the second electrode is preferably designed as an inverted second electrode, wherein preferably the inverted second electrode comprises a sheet-like structure. In particular, the inverted second electrode comprises an opening cut-out in the inverted second electrode, preferably in the center of the inverted second electrode. In connection with the present invention, the terms “opening length” and “opening width” in relation to the term “inverted second electrode” is to be understood as meaning a dimension of the opening cut-out. With other word, the terms “opening length” and “opening width” refer preferably to the length and width of the opening cut-out. Microdischarges occur at edges inside this opening cut-out. The term “length” and “width” in relation to the term “inverted second electrode” is to be understood as meaning a length, in particular a total length, and a width, of the inverted second electrode. The term “thickness” in relation to the term “inverted second electrode” retains its previously introduced meaning as in relation to the “thickness” of the “second electrode”.

An exemplary embodiment of the electrode arrangement is preferred which is characterized in that the first electrode, viewed in the direction of the spacing of the electrodes from each other, has a thickness of at least 10 μm to at the most 50 μm, preferably 35 μm, and/or the second electrode, viewed in the direction of the spacing of the electrodes from each other, has a thickness of at least 10 μm to at most 50 μm, or a thickness of 35 μm, and/or the dielectric element has a thickness of at least 100 μm to at most 300 μm, preferably of at least 220 μm to at most 280 μm, preferably of at least 250 μm to at most 260 μm, and/or the second electrode comprises at least one electrode segment, preferably having a length of 4 cm to 30 cm, wherein preferably two or more electrode segments are arranged parallel or nearly parallel, and/or the ENIG or ENEPIG or ENIPIG or EP or EPIG coating of the second electrode has a thickness of at least 0.3 μm and at most 10 μm, preferably at least 3 μm to at least 7 μm, and/or the second electrode has two or more electrode segments which are movable relative to each other, and/or the second electrode is flexible, such that the second electrode is adaptable to a shape of a surface in contact with the second electrode.

In the context of the present application, details of a thickness of electrodes always refer to a thickness of the electrode without an optionally existing thickness of a coating. In other words, the electrode thickness does not include an optional coating thickness.

In a preferred exemplary embodiment, the ENIG coating of the second electrode has a total thickness of at least 3 μm to at most 8 μm, preferably at least 4 μm to at most 7 μm, most preferably at least 5 μm to at most 6 μm, and a nickel layer of the ENIG coating has preferably a total thickness of at least 2.5 μm to at most 7 μm, preferably at least 4 μm to at most 6 μm, most preferably 5 μm, and a gold layer of the ENIG coating has preferably a total thickness of at least 0.04 μm to at most 0.15 μm, preferably at least 0.08 μm to at most 0.11 μm, most preferably 0.09 μm.

In the context of the present application, details on a thickness of electrodes and/or coatings should preferably be understood taking into account a deviation of plus/minus 10%, preferably 5%, more preferably 1%, due to manufacturing tolerances.

In a preferred exemplary embodiment, the ENEPIG coating of the second electrode has a total thickness of at least 3 μm to at most 8 μm, preferably at least 4 μm to at most 7 μm, most preferably at least 5 μm to at most 6 μm, and a nickel layer of the ENEPIG coating has preferably a total thickness of at least 2.5 μm to at most 7 μm, preferably at least 4 μm to at most 6 μm, most preferably 5 μm, and a palladium layer of the ENEPIG coating has preferably a total thickness of at least 0.05 μm to at most 0.3 μm, preferably at least 0.1 μm to at most 0.25 μm, most preferably 0.175 μm, as well as a gold layer of the ENEPIG coating has preferably a total thickness of at least 0.03 μm to at most 0.1 μm, preferably at least 0.05 μm to at most 0.08 μm, most preferably 0.065 μm.

In a preferred exemplary embodiment, the ENIPIG coating of the second electrode has a total thickness of at least 3 μm to at most 8 μm, preferably at least 4 μm to at most 7 μm, most preferably at least 5 μm to at most 6 μm, and a nickel layer of the ENIPIG coating has preferably a total thickness of at least 2.5 μm to at most 7 μm, preferably at least 4 μm to at most 6 μm, most preferably 5 μm, and a palladium layer of the ENIPIG coating has preferably a total thickness of at least 0.01 μm to at most 0.1 μm, preferably at least 0.04 μm to at most 0.07 μm, most preferably 0.06 μm, as well as a gold layer of the ENIPIG coating has preferably a total thickness of at least 0.03 μm to at most 0.1 μm, preferably at least 0.05 μm to at most 0.08 μm, most preferably 0.07 μm.

In a preferred exemplary embodiment, the EP coating of the second electrode has a total thickness of at least 0.1 μm to at most 0.2 μm, preferably at least 0.14 μm to at most 0.16 μm, most preferably 0.15 μm.

In a preferred exemplary embodiment, the EPIG coating of the second electrode has a total thickness of at least 0.13 μm to at most 0.3 μm, preferably at least 0.18 μm to at most 0.25 μm, most preferably at least 0.2 μm to at most 0.23 μm, and a palladium layer of the EPIG coating has preferably a total thickness of at least 0.1 μm to at most 0.2 μm, preferably at least 0.13 μm to at most 0.17 μm, most preferably 0.15 μm, and a gold layer of the EPIG coating has preferably a total thickness of at least 0.03 μm to at most 0.1 μm, preferably at least 0.05 μm to at most 0.08 μm, most preferably 0.06 μm.

In a preferred exemplary embodiment, the second electrode has a length, preferably a total length, of at least 5 mm to at most 300 mm, preferably at least 25 mm to at most 250 mm, preferably at least 50 mm to at most 200 mm, preferably at least 75 mm to at most 150 mm, preferably at least 100 mm to at most 125 mm.

It is particularly preferred that for mobile application the second electrode has a length, preferably a total length, of at least 5 mm to at most 100 mm, preferably at least 30 mm to at most 70 mm.

It is particularly preferred that for domestic applications the second electrode has a length, preferably a total length, of at least 50 mm to at most 300 mm, preferably at least 100 mm to at most 250 mm.

In a preferred exemplary embodiment, the second electrode has a width, of at least 0.1 mm to at most 2 mm, preferably at least 0.15 mm to at most 1.5 mm, preferably at least 0.2 mm to at most 1 mm, preferably at least 0.25 mm to at most 0.5 mm.

In a preferred exemplary embodiment, the second electrode has a combined width, of at least 0.1 mm to at most 75 mm, preferably at least 1 mm to at most 70 mm, preferably at least 5 mm to at most 60 mm, preferably at least 10 mm to at most 50 mm, preferably at least 15 mm to at most 25 mm.

In a preferred exemplary embodiment the second electrode is designed as an inverted second electrode. It is particular preferred that the inverted second electrode has a width and a length, which overlaps the first electrode by at least 2 mm. Furthermore the inverted second electrode comprises preferably an opening cut-out in the center of the inverted second electrode. The opening cut-out of the inverted second electrode comprise preferably an extension of at least 8 mm smaller than the length of the first electrode and a width of at least 2 mm with a maximum width of at most 8 mm smaller than the width of the first electrode, in particular the total width of the first electrode.

It is particularly preferred that for mobile application the inverted second electrode has a length, preferably a total length, of at least 4 cm to at most 10 cm, preferably at least 6 cm to at most 8 cm.

It is particularly preferred that for domestic application the inverted second electrode has a length, preferably a total length, of at least 10 cm to at most 30 cm, preferably at least 15 cm to at most 20 cm.

In a preferred exemplary embodiment, the inverted second electrode has a width, preferably a total width, of at least 5 mm to at most 10 mm.

An exemplary embodiment of the electrode arrangement is preferred which is characterized in that the first electrode has an Electroless Nickel Immersion Gold (ENIG) coating, or an Electroless Nickel Electroless Palladium Immersion Gold (ENEPIG) coating, or an Electroless Nickel Immersion Palladium Immersion Gold (ENIPIG) coating, or an Electroless Palladium (EP) coating, or an Electroless Palladium Immersion Gold (EPIG) coating.

In a preferred exemplary embodiment, the ENIG coating of the first electrode has a total thickness of at least 3 μm to at most 8 μm, preferably at least 4 μm to at most 7 μm, most preferably at least 5 μm to at most 6 μm, and a nickel layer of the ENIG coating has preferably a total thickness of at least 2.5 μm to at most 7 μm, preferably at least 4 μm to at most 6 μm, most preferably 5 μm, and a gold layer of the ENIG coating has preferably a total thickness of at least 0.04 μm to at most 0.15 μm, preferably at least 0.08 μm to at most 0.11 μm, most preferably 0.09 μm.

In a preferred exemplary embodiment, the ENEPIG coating of the first electrode has a total thickness of at least 3 μm to at most 8 μm, preferably at least 4 μm to at most 7 μm, most preferably at least 5 μm to at most 6 μm, and a nickel layer of the ENEPIG coating has preferably a total thickness of at least 2.5 μm to at most 7 μm, preferably at least 4 μm to at most 6 μm, most preferably 5 μm, and a palladium layer of the ENEPIG coating has preferably a total thickness of at least 0.05 μm to at most 0.3 μm, preferably at least 0.1 μm to at most 0.25 μm, most preferably 0.175 μm, as well as a gold layer of the ENEPIG coating has preferably a total thickness of at least 0.03 μm to at most 0.1 μm, preferably at least 0.05 μm to at most 0.08 μm, most preferably 0.065 μm.

In a preferred exemplary embodiment, the ENIPIG coating of the first electrode has a total thickness of at least 3 μm to at most 8 μm, preferably at least 4 μm to at most 7 μm, most preferably at least 5 μm to at most 6 μm, and a nickel layer of the ENIPIG coating has preferably a total thickness of at least 2.5 μm to at most 7 μm, preferably at least 4 μm to at most 6 μm, most preferably 5 μm, and a palladium layer of the ENIPIG coating has preferably a total thickness of at least 0.01 μm to at most 0.1 μm, preferably at least 0.04 μm to at most 0.07 μm, most preferably 0.06 μm, as well as a gold layer of the ENIPIG coating has preferably a total thickness of at least 0.03 μm to at most 0.1 μm, preferably at least 0.05 μm to at most 0.08 μm, most preferably 0.07 μm.

In a preferred exemplary embodiment, the EP coating of the first electrode has a total thickness of at least 0.1 μm to at most 0.2 μm, preferably at least 0.14 μm to at most 0.16 μm, most preferably 0.15 μm.

In a preferred exemplary embodiment, the EPIG coating of the first electrode has a total thickness of at least 0.13 μm to at most 0.3 μm, preferably at least 0.18 μm to at most 0.25 μm, most preferably at least 0.2 μm to at most 0.23 μm, and a palladium layer of the EPIG coating has preferably a total thickness of at least 0.1 μm to at most 0.2 μm, preferably at least 0.13 μm to at most 0.17 μm, most preferably 0.15 μm, and a gold layer of the EPIG coating has preferably a total thickness of at least 0.03 μm to at most 0.1 μm, preferably at least 0.05 μm to at most 0.08 μm, most preferably 0.06 μm.

In a preferred exemplary embodiment, the first electrode has a length, preferably a total length, of at least 5 mm to at most 300 mm, preferably at least 25 mm to at most 250 mm, preferably at least 50 mm to at most 200 mm, preferably at least 75 mm to at most 150 mm, preferably at least 100 mm to at most 125 mm.

It is particularly preferred that for mobile application the first electrode has a length, preferably a total length, of at least 5 mm to at most 100 mm, preferably at least 30 mm to at most 70 mm.

It is particularly preferred that for domestic application the first electrode has a length, preferably a total length, of at least 50 mm to at most 300 mm, preferably at least 100 mm to at most 250 mm.

In a preferred exemplary embodiment, the first electrode has a width, preferably a total width, of at least 0.1 mm to at most 75 mm, preferably at least 1 mm to at most 70 mm, preferably at least 5 mm to at most 60 mm, preferably at least 10 mm to at most 50 mm, preferably at least 15 mm to at most 25 mm.

It is particularly preferred that for mobile application the first electrode has a width, preferably a total width, of at least 0.1 mm to at most 10 mm, preferably at least 3 mm to at most 7 mm.

It is particularly preferred that for domestic application the first electrode has a width, preferably a total width, of at least 5 mm to at most 30 mm, preferably at least 10 mm to at most 200 mm.

In a preferred exemplary embodiment, the second electrode, which can be brought into contact with a treated surface, a treated porous material or a treated fabric and is electrically connected to a conductive surface, in particular metallic surface, which is in contact with a user of the electrode arrangement, so that the electrode has the same potential as the user. Preferably the second electrode is electrically connected by means of a strap or a loop with a user. As a result, the electrical safety can be significantly increased.

It is therefore apparent that the conductive surface is preferably part of a housing, in which the electrode arrangement is contained.

The lower limit for the thickness of the first electrode is preferably selected to minimize resistive losses.

Therefore, the electrode arrangement is preferably designed pliable, in particular flexible.

The dielectric element is in particular arranged between the first electrode and the second electrode so that, on the one hand, the electrodes are geometrically spaced from each other and, on the other hand, are electrically insulated from each other by the dielectric element. The arrangement is selected in particular so that the first electrode has physical contact with the dielectric element, wherein no air gap is provided between the first electrode and the dielectric element, wherein the second electrode has physical contact with the dielectric element, wherein no air gap is provided between the second electrode and the dielectric element.

The first electrode, the dielectric element and the second electrode preferably thus form a stack, wherein the direction of the spacing of the electrodes from each other by the dielectric element corresponds to a stacking direction. Correspondingly, the aforementioned thicknesses are measured in the stacking direction. A non-thermal plasma is in particular understood to be a plasma in which a temperature describing the distribution of the kinetic energy of the electrons of the plasma, also termed the electron temperature, is not identical and in particular is much higher than a temperature describing the distribution of the kinetic energy of the ions comprised by the plasma, in particular atomic ions or molecular ions, which is also termed the ion temperature. The electron temperature is significantly higher than the ion temperature in the range of a few eV. The ion temperature is rapidly reduced by collisions between ions and the neutral gas or air molecules (the gas or air is only partially ionized, with an overwhelming neutral component remaining). The partially ionized plasma then has a ion temperature, which in particular can be selected within a range of 25° C. (or slightly above room temperature) to at most 100° C. Given the comparatively low ion temperature, such a plasma is also termed a “cold plasma”.

In this context, a plasma designates in particular a material state in which charged particles with opposing charges exist next to each other in the gas phase, wherein averaged over a specific volume, a neutral electric charge results for the considered volume. The plasma also preferably comprises non-charged atoms and molecules that are in electronically, vibrationally or rotationally excited states, and that also are termed excited particles and/or free radicals, overall in particular non-charged reactive atoms and/or molecules that are also termed reactive particles or reactive species.

An exemplary embodiment of the electrode arrangement is preferred which is characterized in that the second electrode possesses a dielectric cover element on a side facing away from the dielectric element that, viewed in the stacking direction, has a thickness of at least 0.2 μm to at most 30 μm. The dielectric cover element serves to protect the second electrode from damage, in particular from chemical or mechanical erosion, and simultaneously prevents direct contact between a treated surface and the second electrode. Given the very thin design of the dielectric cover element, the electrode arrangement is preferably pliable, in particular flexible, even when it possesses the dielectric cover element.

In a preferred exemplary embodiment, the dielectric cover element is designed as a coating, wherein in particular the second electrode is coated with the dielectric cover element.

The electrode arrangement is preferably configured so that the non-thermal plasma, viewed from the dielectric element, is generated on the side of the second electrode, in particular on the surface of the second electrode, or on a surface of the dielectric cover element facing away from the second electrode.

The dielectric cover element serves in particular as a protective coating, in particular for guaranteeing the electrical safety of the electrode arrangement for an operator, and/or for parts treated with the non-thermal plasma.

The ratio of the thickness of the dielectric element to the thickness of the dielectric cover element has to be chosen so that plasma production is enabled, in particular guaranteed, wherein at the same time the breakdown potential of the dielectric element is not exceeded. Measurements with various materials have revealed, in the nature of a guideline, that a ratio of the thickness of the dielectric element to the thickness of the dielectric cover element is preferably at least 10, preferably at least 100, preferably at least 500 to at most 2500.

An exemplary embodiment of the electrode arrangement is preferred that is characterized in that the first electrode has a dielectric base element on a side facing away from the dielectric element. With the assistance of the dielectric base element, it is preferably possible to prevent or suppress corona discharges proceeding from the first electrode that would otherwise reduce efficiency of the electrode arrangement. Furthermore, the dielectric base element reduces the risk of creepage currents and increases safety, in particular electrical safety. A thickness of the dielectric base element measured in the stack direction is preferably chosen such that, on the one hand, the corona discharges that proceed from the first electrode are effectively and efficiently, preferably completely suppressed, wherein on the other hand, the electrode arrangement remains pliable overall, in particular flexible. It is in particular possible for the dielectric base element to possess a thickness of at least 1 μm to at most 250 μm, preferably at most 30 μm.

The dielectric base element is preferably designed flat and extends preferably along an overall extension of the first electrode. In a preferred exemplary embodiment, the dielectric base element is designed as a coating, wherein the first electrode is coated with the dielectric base element, in particular on its side facing away from the dielectric element.

An exemplary embodiment of the electrode arrangement is preferred that is characterized in that the at least one electrode, selected from the first electrode and the second electrode, has a material or consists of a material that is selected from a group consisting of copper, silver, gold, iron and aluminum. Other conductive materials, in particular metals or metalloids can also be used for the at least one electrode. It is also possible to produce at least one of the first and second electrodes from a metal alloy, in particular an alloy that has at least one of the aforementioned elements. It is important for the electrodes to comprise or consist of a conductive material, preferably with minimal electrical resistance.

An exemplary embodiment of the electrode arrangement is also preferred that is characterized in that at least one element selected from the dielectric element, dielectric cover element and dielectric base element has a material or consists of a material, wherein the material is selected from the group consisting of silicon nitride (SiN), a silicate, in particular quartz (SiO2), a thermosetting compound, a non-conductive compound, a glass, and a plastic, in particular polyamide. Other inorganic or organic materials are also possible for the at least one element. It is important for the selected material to have dielectric properties and in particular be designed nonconductive, in particular as an electrical insulator.

An exemplary embodiment of the electrode arrangement is also preferred that is characterized in that the dielectric element has a material or consists of a material, wherein the material is selected from the group of woven glass reinforced hydrocarbon ceramic composites, in particular consisting of Rogers 4350B.

With respect to specific products, in particular branded products, such as Rogers 4350B, this description is preferably understood to mean a product and/or material which could be obtained from the manufacturer and/or retailer of the product on the date establishing the priority of the present application.

An exemplary embodiment of the electrode arrangement is preferred which is characterized in that the first electrode is designed flat. Particularly preferably, the first electrode is designed as a layer electrode or leaf electrode. This enables a homogeneous distribution of the generated plasma, and a particularly homogeneous distribution of the output as well as reducing unwanted stray capacitance.

One preferred exemplary embodiment provides that the second electrode is designed structured. The second electrode can in particular be tailored in terms of its geometric structure to specific requirements for the electrode arrangement, in particular to a specific use of the electrode arrangement.

One preferred exemplary embodiment of the electrode arrangement provides that the second electrode has a comb-like structure. In particular, this designates a structure in which the second electrode has a backbone element, preferably an elongated backbone element, proceeding from which, preferably a number of, electrode branches extend that are oriented parallel to each other and electrically connected to the backbone element. It is also possible for the second electrode to have a linear structure, wherein the linear structure has at least one straight line. Preferably, the linear structure has a plurality of lines that are in particular arranged parallel to each other and electrically connected to each other.

It is also possible for the second electrode to have a winding structure, in particular a wavy structure. It is in particular possible for the second electrode to be designed as a wavy line. It can also be preferably provided that the second electrode has a plurality of wavy lines arranged parallel to each other that are electrically connected to each other. In particular, a comb-like structure is also possible in which electrode branches, proceeding from a backbone element, extend in the form of wavy lines and are electrically connected to the backbone element and preferably oriented parallel to each other.

It is also possible for the second electrode to have a spiral structure, in particular in the form of a circular spiral or angular spiral, or for the second electrode to have a meandering structure.

If the second electrode has a line or plurality of lines, for example in the form of a wavy line or a straight line, or in the form of a spiral or meandering line, such a line of the second electrode preferably has a width of at least 250 μm to at most 1000 μm. Such a width has proven to be particularly beneficial on the one hand with regard to the electrical properties of the electrical arrangement and the properties of the generated plasma and, on the other hand, with regard to the pliability and flexibility of the electrode arrangement.

A preferred exemplary embodiment provides that the second electrode has a flat structure with at least one recess, which is also known as the inverted second electrode. This means in particular that the second electrode is designed flat, wherein it has at least one recess, preferably at least one penetration, in particular in its surface. In this case, surface microdischarges can be generated, in particular in the at least one recess. The surface microdischarges are generated in particular at edges of the at least one recess.

It was revealed from tests and simulation studies that edges of the at least one recess preferably have a distance relative to each other of at least 0.5 mm, and preferably more than 0.5 mm. In particular, the at least one recess preferably has a width of at least 0.5 mm, preferably more than 0.5 mm.

Numeric simulations of the electrode arrangement and extensive experimental studies have shown that the electrical field at the edges of such a recess in the second electrode has an extension of approximately 30 μm to 50 μm. It is this electric field that initiates the microdischarges. At two opposing edges of such a recess, microdischarges can therefore arise in two regions with an extension perpendicular to the edge of approximately 1 mm in each case. To prevent interference amongst the discharges, a spacing between the edges must therefore be chosen that is much greater than twice the extension of the individual microdischarges, i.e., greater than 2 mm.

The at least one recess is preferably designed like an engraved structure in the second electrode.

According to a development of the invention, the first electrode, perpendicular to the stack direction, projects at least sectionally, preferably entirely, beyond a margin of the second electrode. In this case, surfaces can generate microdischarges on an outer edge as well, i.e., an outer peripheral line or edge of the second electrode.

Alternatively or in addition, it is possible for the first electrode, viewed perpendicular to the stack direction, to have, at least sectionally, a lesser extension than the second electrode so that the second electrode projects at least sectionally, preferably entirely in this direction beyond the first electrode. In this case, at least where the second electrode projects laterally beyond the first electrode, i.e., perpendicular to the stack direction, it is possible for no microdischarges to be generated at the peripheral edge of the second electrode.

Viewed perpendicular to the stack direction, the dielectric element preferably extends beyond the outer peripheral lines of both electrodes, i.e., of the first electrode and second electrode.

It is moreover possible for the second electrode to be arranged on the dielectric element, in particular to be deposited thereupon, for example by physical vapor deposition, etc. It is however also possible for the second electrode to be embedded in the dielectric element.

It is moreover possible for the first electrode to be arranged under the dielectric element, in particular to be deposited underneath, for example by physical vapor deposition, etc. It is however also possible for the first electrode to be embedded in the dielectric element.

Alternatively or in addition, the dielectric cover element is preferably designed flat, wherein it preferably entirely houses or encapsulates the second electrode and thus protects it from contact with a surface to be treated as well as from damage, in particular chemical or mechanical erosion.

Alternatively or in addition, the dielectric base element is preferably designed flat, and thus protects in particular one of the two sides of the first electrode facing away from the second electrode from external influences.

The object is also achieved in that a plasma source is created for generating a non-thermal plasma that has a voltage source and an electrode arrangement according to one of the above-described exemplary embodiments. The voltage source is electrically connected to at least the first electrode. In conjunction with a plasma source, the advantages result that were explained with regard to the electrode arrangement.

Since the voltage source is electrically connected to at least the first electrode, it means in particular that an electric voltage or an electric signal, in particular an AC voltage is applied to the first electrode.

It is preferably provided that the second electrode is earthed or is grounded. This increases the electrical safety of the plasma source since the second electrode is arranged closer to a surface to be treated than the first electrode. If for example a break occurs when bending the flexible electrode arrangement and part of the second electrode exits the dielectric cover element, there is no danger from this part of the second electrode contacting the treated surface, in particular the skin of the patient, when the second electrode is earthed or grounded.

In a preferred exemplary embodiment of the plasma source, it is provided that the voltage source is also electrically connected to the second electrode. Particularly preferably, the second electrode is grounded or earthed via the voltage source.

Alternatively, it is also possible for the voltage source to only be electrically connected to the first electrode, wherein preferably the voltage source can be connected on the one hand and the second electric source can be connected on the other hand to a common earth point or common ground point.

An exemplary embodiment of the plasma source is preferred that is characterized in that an AC voltage can be applied to the first electrode, wherein the second electrode is earthed. As mentioned above, this can be done through the voltage source, or outside of the voltage source by contacting the second electrode to an earth or ground point.

An exemplary embodiment of the plasma source is also possible in which parts of the second electrode do not have a specific electric potential and thus floats.

An exemplary embodiment of the plasma source is preferred which is characterized in that the plasma source, preferably the voltage source, is configured to generate an AC voltage that has an amplitude of at least 0.5 kV from peak to peak which is also termed kVpp, to at most 5 kVpp, preferably from at least 1 kVpp to at most 4.5 kVpp, preferably from at least 1.5 kVpp to at most 4 kVpp, and/or at a frequency of at least 10 kHz to at most 100 kHz, preferably from at least 20 kHz to at most 80 kHz, preferably from at least 30 kHz to at most 60 kHz, preferably from at least 40 kHz to at most 50 kHz, preferably 50 kHz. Given the very thin design of the electrode arrangement, it is possible to apply an AC voltage that has a comparatively low amplitude to the first electrode. This increases the electrical safety of the plasma surface and minimizes a safe distance to be maintained between a surface to be treated and the surface of the electrode arrangement at which the plasma is generated.

An exemplary embodiment of the plasma source is preferred which is characterized in that the plasma source, preferably the voltage source, is configured to generate an AC power, in particular a plasma power, that has a range of at least 0.1 Watt/cm to 1.0 Watt/cm for the length of the second electrode. The power level translates directly into the electron flux, which is needed to refresh the fabrics and destroy the malodour molecules contained in the fabric by electron-impact dissociation.

An exemplary embodiment of the plasma source is preferred which is characterized in that the plasma source, preferably the voltage source, is configured to provide a, preferably total, electrical plasma power of at least 0.5 W to at most 5 W, preferably from at least 1.0 W to at most 3 W, preferably from at least 1 W to at most 2 W, for mobile application.

An exemplary embodiment of the plasma source is preferred which is characterized in that the plasma source, preferably the voltage source, is configured to provide a, preferably total, electrical plasma power of at least 5 W to at most 50 W, preferably from at least 10 W to at most 40 W, preferably from at least 15 W to at most 30 W, for domestic application.

An exemplary embodiment of the plasma source is preferred which is characterized in that the plasma source, preferably the voltage source, is configured to provide an electrical power of at least 0.1 watt to at most 1 watt per cm length of the electrode assembly which is also termed W/cm, preferably from at least 0.2 W/cm to at most 0.4 W/cm. For the domestic application and the mobile application the same range of power levels/cm applies.

The very thin geometric design of the electrode embodiment makes it possible to additionally or alternately select a frequency for the AC voltage that lies above a maximum audio frequency, i.e., in particular above 20 kHz. This is also especially promoted because the first electrode is preferably designed flat or as a layer electrode or leaf electrode, which minimizes the leakage capacity of the electrode arrangement. The choice of such high frequencies above the maximum audio frequency increases the electrical safety of the plasma source on the one hand, and on the other hand piezoamplifiers can be used, in particular when a frequency of at least 50 kHz is chosen. This in turn permits a further miniaturization of the plasma source because such piezoamplifiers can be designed very small. In particular, such a piezoamplifier can have a thickness of approximately 2 mm, a width of approximately 8 mm, and a length of approximately 50 mm. It is accordingly possible to provide a plasma source that for example has the size of a pencil. In the final analysis, the plasma source per se is no longer restrictive to the size of a plasma device that has the plasma source; instead, it has the size of an electrical storage device for supplying the voltage source with electrical power if it is a portable, mains-independent device. In particular, the size of the battery is restrictive for the possible miniaturization of the corresponding device.

In particular, an exemplary embodiment of the plasma source is preferred that is characterized in that the plasma source, in particular the voltage source, has a piezoamplifier. This yields the above-described advantage of a smaller design and improved options for miniaturizing the plasma source.

Such piezoamplifiers or piezotransformers can be operated optimally at a frequency of about 50 kHz since their resonance frequency lies within this range. Moreover, they are optimally operated at a voltage of at most 3 kVpp. Both enable the electrode arrangement proposed here given their low electrical capacitance which prevents otherwise high loss, and given the possibility of operating them at a comparatively low AC voltage amplitude.

Alternatively or in addition an exemplary embodiment of the plasma source is preferred that is characterized in that the plasma source, in particular the voltage source, has a tesla coil or a resonant transformer or a resonant transformer in combination with a coil transformer.

In particular, an exemplary embodiment of the plasma source is preferred that is characterized in that the plasma source has a tesla coil or a resonant transformer or a resonant transformer in combination with a coil transformer as the voltage source or electrically arranged between and in electrical contact with the voltage source and the first electrode for amplifying an AC voltage applied to the first electrode.

The object is lastly achieved by creating a method for operating a plasma source, in particular for deactivation/destroying/removing of undesirable and/or harmful substances associated with a material to be treated, wherein within the context of the method, electrical voltage is applied to the electrode arrangement according to one of the above-described exemplary embodiments by means of a voltage source, and/or wherein a plasma source is operated according to one of the above-described exemplary embodiments. In conjunction with the method, the advantages are realized in particular that were already explained in conjunction with the electrode arrangement and/or with the plasma source. In particular it is preferred, that the second electrode is arranged near or in contact with a fabric or a surface to be treated.

In particular, surface microdischarges are generated in the context of the method. By varying the electrical parameters of the plasma source, a plasma chemistry of the plasma generated by the surface microdischarges can be modified.

An embodiment of a method is preferred that is characterized in that an AC voltage with an amplitude of at least 0.5 kVpp to at most 5 kVpp, preferably from at least 1 kVpp to at most 4.5 kVpp, preferably from at least 1.5 kVpp to at most 4 kVpp is generated. In particular, this yields the advantage of increased electrical safety while operating the plasma source.

An embodiment of the method is also preferred in which the AC voltage is generated at a frequency of at least 10 kHz to at most 100 kHz, preferably from at least 20 kHz to at most 80 kHz, preferably from at least 30 kHz to at most 60 kHz, preferably from at least 40 kHz to at most 50 kHz, preferably 50 kHz. On the one hand, this yields the advantage of increased electrical safety when operating the plasma source, and on the other hand the advantage that piezoamplifiers can be used which in turn enables a further miniaturization of the plasma source.

A device for the plasma treatment of an object, in particular a surface that has an electrode arrangement and/or plasma surface according to one of the above-described exemplary embodiments, is also part of the invention.

Moreover, the invention also includes use of an electrode arrangement and/or a plasma source for deactivation of odor-relevant molecules associated with a material to be treated, the deactivation of allergens, bacteria, fungi or mites (especially with respect to a domestic device).

The first electrode and the second electrode are preferably fully encapsulated in nonconductive and/or dielectric material which increases the safety of the electrode arrangement.

Overall, the following advantages also result in conjunction with the electrode arrangement, plasma source and method:

Due to the very thin design of the electrode arrangement, it can be bent very easily so that a flexible design in the production of devices that have the electrode arrangement and/or the plasma source is possible.

A small voltage source can be used which permits a reduction of the size of the amplifier.

Moreover, greater conversion efficiency of the output of the voltage source to the operating voltage is possible, for example when converting a DC voltage to an AC voltage.

Due to the greater electrical safety, it is possible to reduce the thickness of an insulator material in a housing of the electrode arrangement where the plasma source is so that smaller safe distances are possible (creepage current constraint).

In the event of an accident or a malfunction, the risk of an electric shock to the operator of the electrode arrangement or the plasma source, and to the treated surface, in particular the skin of the user, is minimized. The maximum electrical current is determined by the voltage and the resistance used. Furthermore, the electrode arrangement or the plasma source is provided with interrupts and/or shut-down during current surges and with a protection against leakage currents or creepage currents.

Given the increased electrical safety and accordingly reduced safety requirements, in particular greater design freedom in the structure of the electrode arrangement is possible.

The invention will be further explained below with reference to the drawing. In the drawing:

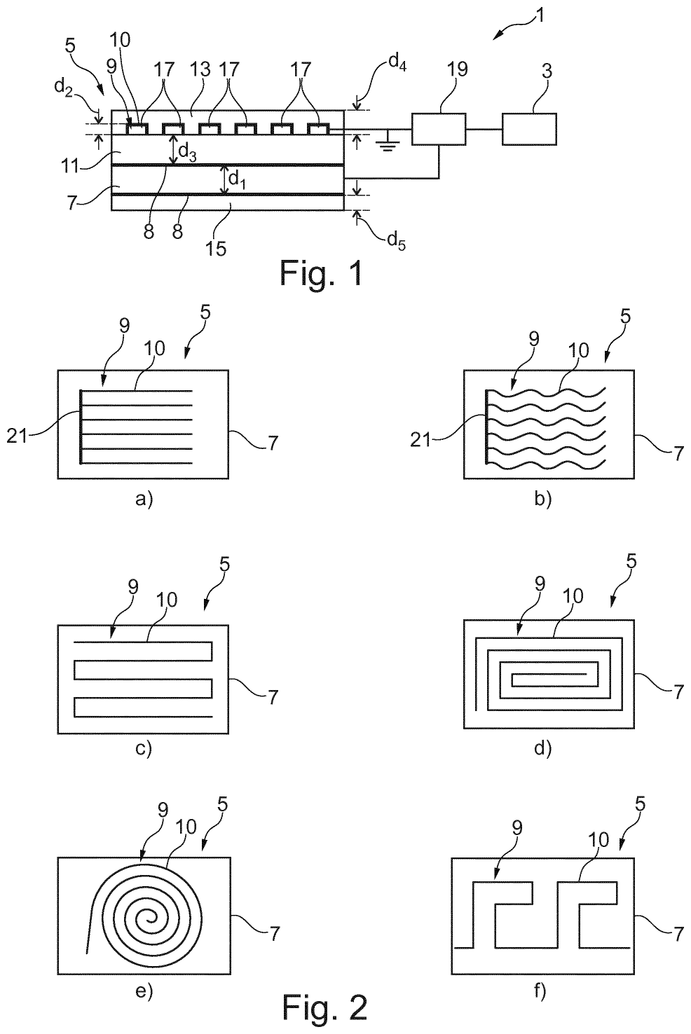

FIG. 1 shows a schematic representation of an exemplary embodiment of a plasma source;

FIG. 2 shows a plurality of different exemplary embodiments of an electrode arrangement with respect to a structure of the second electrode;

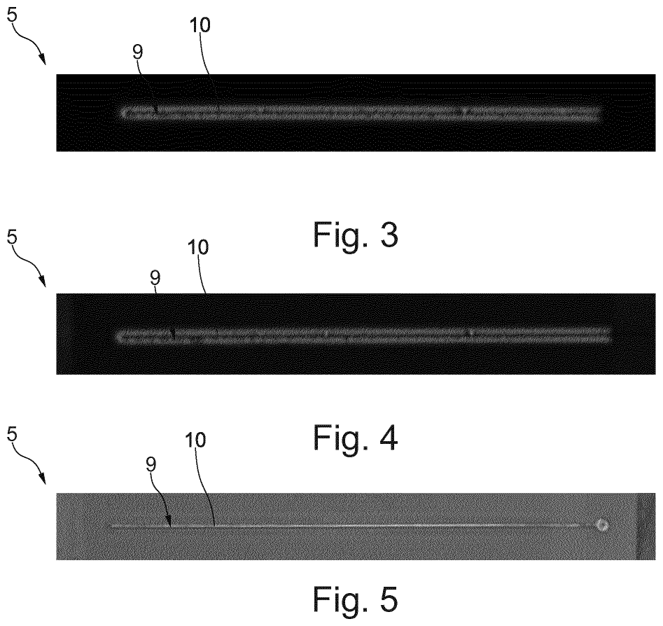

FIG. 3 shows an image of an electrode arrangement according to the invention;

FIG. 4 shows an image of the electrode arrangement according to FIG. 3 after 18 hours of plasma operation, and

FIG. 5 shows an image of an electrode arrangement according to FIG. 3 or 4 without plasma operation.

FIG. 1 shows a schematic representation of an exemplary embodiment of a plasma source 1 that is configured to generate a non-thermal plasma. The plasma source 1 has a voltage source 3 that is electrically connected to an electrode arrangement 5. For its part, the electrode arrangement 5 is configured to generate a non-thermal plasma.

The electrode arrangement 5 has a first electrode 7 and a second electrode 9, wherein between the first electrode 7 and the second electrode 9, a dielectric element 11 is arranged so that the two electrodes 7, 9 are electrically insulated from each other and spaced from each other by the dielectric element 11. The two electrodes 7, 9 and the dielectric element 11 form a stack, wherein viewed in the stacking direction, the dielectric element 11 is arranged on the first electrode 7, and the second electrode 9 is arranged on the dielectric element 11.

Viewed in the stacking direction, the first electrode 7 has preferably a first thickness d1 of at least 10 μm, wherein the second electrode 9, also viewed in the stacking direction, has preferably a second thickness d2 of at least 10 to at most 50 μm. Viewed in the stacking direction, the dielectric element 11 has a third thickness d3 of at least 100 μm to at most 300 μm.

The second electrode 9 has an Electroless Nickel Immersion Gold (ENIG) coating 10, or an Electroless Nickel Electroless Palladium Immersion Gold (ENEPIG) coating 10, or an Electroless Nickel Immersion Palladium Immersion Gold (ENIPIG) coating 10, or an Electroless Palladium (EP) coating 10, or an Electroless Palladium Immersion Gold (EPIG) coating 10, and/or the dielectric element 11 is made of a woven glass reinforced hydrocarbon ceramic.

The electrode arrangement 5 is accordingly designed as a thin layer electrode arrangement and has a very slight thickness overall. This renders it pliable overall so that it can be flexibly adapted to a plurality of different uses, and in particular to a plurality of geometrically different surfaces to be treated. Moreover, the electrode arrangement 5 can be operated at a low voltage, in particular at less than 5 kVpp, due to its very thin design which increases the electrical safety of the plasma source 1.

The first electrode 7 has an Electroless Nickel Immersion Gold (ENIG) coating 8, or an Electroless Nickel Electroless Palladium Immersion Gold (ENEPIG) coating 8, or an Electroless Nickel Immersion Palladium Immersion Gold (ENIPIG) coating 8, or an Electroless Palladium (EP) coating 8, or an Electroless Palladium Immersion Gold (EPIG) coating 8.

The second electrode 9 has a dielectric cover element 13 on a side facing away from the dielectric element 11 that has a fourth thickness d4 of at least 0.1 μm to at most 30 μm viewed in the stacking direction.

The dielectric cover element 13 is preferably designed as a coating, wherein in particular the second electrode 9 is coated with the dielectric cover element 13, or with the material of the dielectric cover element 13. The dielectric cover element 13 thereby covers the second electrode 9 preferably completely.

The first electrode 7 has a dielectric base element 15 on the side facing away from the dielectric element 11. This is advantageously designed flat and extends along an overall extension of the first electrode 7 and therefore entirely covers it, at the bottom in FIG. 1. Consequently, the dielectric base element 15 very efficiently prevents coronal discharges that could otherwise proceed from the first electrode 7 so that the efficiency of the electrode arrangement 5 is increased by the electric base element 15. A fifth thickness d5 of the dielectric base element 15 is preferably selected so that on the one hand coronal discharges emitted by the first electrode 7 are reliably avoided, wherein on the other hand, the electrode arrangement 5 is designed pliable overall.

Overall, a stacked electrode arrangement 5 results with the following stack sequence: On the dielectric base element 15, the first electrode 7 is arranged on which the dielectric element 11 is arranged. The second electrode 9, on which the dielectric cover element 13 is arranged, is arranged thereupon.

The at least one first electrode 7 and/or the at least one second electrode 9 preferably has/have a material that is selected from a group consisting of copper, silver, gold and aluminum. Preferably, at least one of the first and second electrodes 7, 9 consist of the aforementioned materials.

Other conductive materials are possible for the electrodes 7, 9, in particular alloys as well, particularly preferably based on at least one of the aforementioned elements.

The dielectric cover element 13 and/or the dielectric base element 15 preferably has/have a material that is selected from the group consisting of silicon nitride, a silicate, in particular quartz, a glass, and a plastic, in particular polyamide. It is also possible for at least one of the aforementioned elements to consist of one of the aforementioned materials. Other inorganic or organic materials are also possible for the aforementioned elements as long as they have dielectric and in particular electrically insulating properties.

The first electrode 7 is preferably designed flat, in particular as a layer or leaf electrode.

The second electrode 9 is preferably designed structured. In particular, in the exemplary embodiment shown in FIG. 1, it has a plurality of linear partial electrodes 17. The structure of the second electrode 9 can in particular be tailored to a specifically designed use of the electrode arrangement 5.

The voltage source 3 is in particular electrically connected to the first electrode 7, wherein an AC voltage can be applied to the first electrode 7. The second electrode 9 is preferably earthed or grounded. In the exemplary embodiment described here, both electrodes 7, 9 are electrically connected by an amplifier 19 to the voltage source 3. The amplifier 19 is preferably designed as a piezoamplifier.

The electrode arrangement 5 is preferably operated with an AC voltage with an amplitude that is at least 0.5 kVpp to at most 5 kVpp, preferably from at least 1 kVpp to at most 4.5 kVpp, preferably from at least 1.5 kVpp to at most 4 kVpp. The AC voltage preferably has a frequency of at least 10 kHz to at most 100 kHz, preferably from at least 20 kHz to at most 80 kHz, preferably from at least 30 kHz to at most 60 kHz, preferably from at least 40 kHz to at most 50 kHz, preferably 50 kHz.

FIG. 2 shows a plurality of different exemplary embodiments of the electrode arrangement 5, wherein the first flat electrode 7 and the structured second electrode 9 are schematically portrayed in a plan view. Furthermore, the second electrode 9 shown in FIG. 2a) to FIG. 2f) have all an Electroless Nickel Immersion Gold (ENIG) coating 10, or an Electroless Nickel Electroless Palladium Immersion Gold (ENEPIG) coating 10, or an Electroless Nickel Immersion Palladium Immersion Gold (ENIPIG) coating 10, or an Electroless Palladium (EP) coating 10, or an Electroless Palladium Immersion Gold (EPIG) coating 10.

A second electrode 9 is portrayed in FIG. 2a) that has a comb-like structure with a plurality of straight lines that are arranged parallel to each other, are electrically connected to each other, and extend to the right proceeding from common backbone element 21 in FIG. 2a).

In FIG. 2b), the second electrode 9 also has a comb-like structure, wherein serpentine partial electrodes extend parallel to each other proceeding from the common backbone element 21. The individual partial electrodes are electrically connected to each other by the common backbone element 21.

In FIG. 2c), the second electrode 9 also has a linear structure, however in the form of a path running as an angular zigzag line.

In FIG. 2d), the second electrode 9 has the shape of an angular spiral.

In FIG. 2e), the second electrode 9 has the shape of a round spiral, in particular a circular spiral. Finally, the second electrode 9 in FIG. 2f) has a meandering structure.

Furthermore, the invention is explained by the following experimental tests:

FIG. 3 shows an image of an electrode arrangement 5 according to the invention, in particular the initial plasma emission at the start of a long term test. The second electrode 9 is formed as a straight line and consists of copper and has, viewed in the stacking direction, a thickness of 35 μm. Furthermore the second electrode 9 has an ENIG coating 10 with a thickness of the nickel layer of at least 3 to at most 6 μm and a thickness of the gold layer of at least 0.05 μm to at most 0.1 μm. The dielectric element 11 consists of Rogers 4350B and has a thickness of 254 μm.

FIG. 4 shows an image of the electrode arrangement 5 according to FIG. 3 after 18 hours of plasma operation.

FIG. 5 shows an image of an electrode arrangement 5 according to FIG. 3 or 4 without plasma operation. Only minor signs of change in appearance are visible, which do not affect the operability of the electrode arrangement 5. For typical odor removal applications of 1 minute each, the electrode arrangement 5 remains in good conditions even after more than 1000 applications or at least 3 years of use.

1. An electrode arrangement for generating a non-thermal plasma,
comprising:
a first electrode and a second electrode, wherein
the first electrode and the second electrode are electrically insulated from each other and spaced from each other by a dielectric element,
characterized in that:
the second electrode comprises an Electroless Nickel Immersion Gold (ENIG) coating, an Electroless Nickel Electroless Palladium Immersion Gold (ENEPIG) coating, an Electroless Nickel Immersion Palladium Immersion Gold (ENIPIG) coating, an Electroless Palladium (EP) coating, or an Electroless Palladium Immersion Gold (EPIG) coating, or
the dielectric element comprises a woven glass reinforced hydrocarbon ceramic.

2. The electrode arrangement according to claim 1, characterized in that:
the first electrode, viewed in a direction towards the second electrode, comprises a thickness of at least 10 μm to at most 50 μm, preferably 35 μm, or
the second electrode, viewed in a direction towards the first electrode, comprises a thickness of at least 10 μm to at most 50 μm or
the dielectric element comprises a thickness of at least 100 μm to at most 300 μm or
the second electrode comprises at least one electrode segment comprising a length of 4 to 30 cm, wherein two or more electrode segments are arranged in parallel or near-parallel, and/or
the ENIG, or ENEPIG, or ENIPIG or EP or EPIG coating of the second electrode has a thickness of at least 0.3 to at most 10 μm or
the second electrode has two or more electrode segments which are movable relative to each other, and/or the second electrode is flexible, so that the second electrode is adaptable to a shape of a surface in contact with the second electrode.

3. The electrode arrangement according to claim 1, characterized in that a dielectric cover element is arranged on a side of the second electrode facing away from the dielectric element, wherein the cover element, viewed in the stacking direction of the electrode, comprises a thickness of at least 0.2 μm to at most 30 μm.

4. The electrode arrangement according to claim 1, characterized in that a dielectric base element is arranged on a side of the first electrode facing away from the dielectric element.

5. The electrode arrangement according to claim 1, characterized in that at least one electrode, selected from the first electrode and the second electrode, comprises a material or consists of a material that is selected from a group consisting of copper, silver, gold and aluminium.

6. The electrode arrangement according to claim 1, characterized in that at least one element, selected from the dielectric cover element and the dielectric base element, comprises a material or consists of a material that is selected from a group consisting of silicon nitride, a silicate, in particular quartz, a glass, and a plastic.

7. The electrode arrangement according to claim 1, characterized in that the first electrode is designed flat, or the second electrode is designed structured.

8. The electrode arrangement according to claim 1, characterized in that the second electrode comprises a comb-like structure, a linear structure with at least one imaginary line, a winding structure, a spiral structure, a meandering structure, or a flat structure with at least one recess.

9. A plasma source for generating a non-thermal plasma, comprising a voltage source and an electrode arrangement according to claim 1, wherein the voltage source is electrically connected at least to the first electrode.

10. The plasma source according to claim 9, characterized in that the voltage source is adapted to apply an AC voltage to the first electrode, wherein the second electrode is preferably earthed or grounded.

11. The plasma source according to claim 9, characterized in that the plasma source is configured to generate AC voltage with an amplitude of at least 0.5 kVpp to at most 5 kVpp, or at a frequency of at least 10 kHz to at most 100 kHz.

12. The plasma source according to claim 9, characterized in that the plasma source comprises a piezoamplifier as the voltage source or electrically arranged between and in electrical contact with the voltage source and the first electrode for amplifying an AC voltage applied to the first electrode.

13. The plasma source according to claim 9, characterized in that the plasma source has a tesla coil or a resonant transformer or a resonant transformer in combination with a coil transformer as the voltage source or electrically arranged between and in electrical contact with the voltage source and the first electrode for amplifying an AC voltage applied to the first electrode.

14. The plasma source according to claim 9, characterized in that the voltage source is configured to provide an electrical power of at least 0.1 watt to at most 1 watt per cm length of the electrode assembly.

15. A method for removing of undesirable or harmful substances associated with a material to be treated, wherein an electrical voltage is applied to an electrode arrangement according to claim 1 by means of a voltage source.

16. The method according to claim 15, characterized in that the plasma source is operated with an alternating current (AC) voltage comprising an amplitude of at least 0.5 kvpp to at most 5 kvpp, or at a frequency of at least 10 khz to at most 100 khz.

17. The electrode arrangement according to claim 1, characterized in that the second electrode comprises a plurality of straight line elements arranged parallel to one another and electrically connected to one another.

18. The electrode arrangement according to claim 1, characterized in that the first electrode comprises a sheet-like structure.

19. The electrode arrangement according to claim 2, wherein the dielectric element comprises a thickness of at least 100 μm to at most 300 μm.

20. The plasma source according to claim 9, characterized in that the plasma source is configured to generate AC voltage with an amplitude of at least 1.5 kVpp to at most 4 kVpp.
